(19) United States
(12) Patent Application Publication
(10) Pub. No.: US 2026/0207129 A1
(43) Pub. Date: Jul. 23, 2026

(54) METHOD AND SYSTEM FOR CAMERA SLEEP ENDOSCOPY

(71) Applicant: Northwestern University, Evanston, IL (US)

(72) Inventors: Claus-Peter Richter, Skokie, IL (US); Joaquin Cury, Chicago, IL (US)

(21) Appl. No.: 19/192,763

(22) Filed: Apr. 29, 2025

Related U.S. Application Data
(60) Provisional application No. 63/642,208, filed on May 3, 2024.

(51) Int. Cl. A61B 5/00; A61B 1/005; A61B 1/06; A61B 1/233; A61B 17/00

(52) U.S. Cl. CPC A61B 5/4818; A61B 1/0052; A61B 1/0684; A61B 1/233; A61B 5/4836; A61B 17/00; A61B 2217/007

(57) ABSTRACT

A method of diagnosing obstructive sleep apnea includes mounting a camera in a flexible probe that is sized to fit within a nasal cavity of a patient. The method also includes capturing, by the camera mounted within the probe, images of the patient while the patient is sleeping. The method further includes analyzing, by a computing system in communication with the camera, the captured images to determine whether sleep apnea is present.

### CROSS-REFERENCE TO RELATED APPLICATION

The present application claims the priority benefit of U.S. Provisional Patent App. No. 63/642,208 filed on May 3, 2024, the entire disclosure of which is incorporated by reference herein.

### REFERENCE TO GOVERNMENT RIGHTS

This invention was made with government support under grant numbers HL119810 and DC018666 awarded by the National Institutes of Health. The government has certain rights in the invention.

### BACKGROUND

Obstructive sleep apnea (OSA) is the most common type of sleep apnea and is caused by complete or partial cessation of breathing due to obstructions of the upper airway. OSA can occur when the airway at the throat collapses. Such collapsing occurs because the airway at the throat is composed of soft tissue, and this soft tissue relaxes during sleep, resulting in the collapse. OSA is characterized by repetitive episodes of shallow or paused breathing during sleep despite the effort to breathe. Additionally, OSA is usually associated with a reduction in blood oxygen. Individuals with OSA are rarely aware of difficulty breathing, even upon awakening. Rather, OSA is often recognized as a problem by others who observe the individual during episodes or is suspected because of its effects on the body. As a result, symptoms may be present for years or even decades without identification. During this time, the individual may become conditioned to the daytime sleepiness and fatigue associated with significant sleep disturbances. Individuals who generally sleep alone are often unaware of the condition, without a regular bedpartner to notice and make them aware of their symptoms. Persons with OSA have a 30% higher risk of heart attack or death than those unaffected.

### SUMMARY

An illustrative method of diagnosing obstructive sleep apnea includes mounting a camera in a flexible probe that is sized to fit within a nasal cavity of a patient. The method also includes capturing, by the camera mounted within the probe, images of the patient while the patient is sleeping. The method further includes analyzing, by a computing system in communication with the camera, the captured images to determine whether sleep apnea is present.

In one embodiment, the flexible probe includes a stabilization wire that bends to conform the flexible probe to the nasal cavity of the patient. In another embodiment, the method further includes analyzing the captured images to identify a location of the sleep apnea. In another embodiment, the camera is mounted in an imaging port of the flexible probe, the flexible probe also includes an irrigation port, and the method further includes controlling, by the computing system, an irrigation source that is connected to the irrigation port. In one embodiment, an end of the irrigation port is angled toward the imaging port, and the method includes controlling the irrigation source to clean the camera.

In another embodiment, the camera is mounted in an imaging port of the flexible probe, the flexible probe also includes a vacuum port, and the method includes controlling, by the computing system, a vacuum source that is connected to the vacuum port. In another embodiment, controlling the vacuum source comprises activating the vacuum source to remove debris from an end of the flexible probe such that the debris flows through the vacuum port. The method can also include activating, by the computing system, a light source for the camera to provide illumination such that the images can be captured. In another embodiment, the flexible probe is positioned in in an upper pharynx of the patient to monitor an upper airway of the patient. In one embodiment, the flexible probe comprises a feeding tube. In another embodiment, the method includes determining, by the computing system, whether an obstruction relating to the sleep apnea occurs during inhalation or during exhalation.

An illustrative system for use in diagnosis of obstructive sleep apnea includes a probe that includes an imaging port. The system also includes a camera sized to fit into the imaging port. The system further includes an illumination source sized to fit into the imaging port alongside the camera. An outer diameter of the probe is sized to fit within a nostril of a patient such that the camera captures images of a portion of an airway of the patient.

In one embodiment, the probe further includes an irrigation port that connects to an irrigation source. In another embodiment, the irrigation port includes a universal connector that attaches to the irrigation source. In another embodiment, the universal connector comprises a valve that controls the irrigation source. In one embodiment, a distal end of the irrigation port is angled toward a distal end of the imaging port. In another embodiment, a distal end of the imaging port is flush with a distal end of the probe, and a distal end of the irrigation port extends past the distal end of the probe.

In another embodiment, the probe further includes a vacuum port that connects to a vacuum source. In one embodiment, the vacuum port includes a universal connector that connects to the vacuum source. In another embodiment, the illumination source comprises a plurality of light-emitting diodes. In one embodiment, the system includes a stabilization wire within the probe, where the stabilization wire maintains the probe in a desired shape.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts a schematic of the obstructive sleep apnea diagnosis system in accordance with an illustrative embodiment.

FIG. 2A is an end view of a probe for performing obstructive sleep apnea diagnosis in accordance with an illustrative embodiment.

FIG. 2B is a cross-sectional side view of the probe for diagnosing obstructive sleep apnea in accordance with an illustrative embodiment.

FIG. 3 is a block diagram of a computing system to implement the sleep apnea monitoring system in accordance with an illustrative embodiment.

### DETAILED DESCRIPTION

Snoring, hypopnea, and, in its severest form, obstructive sleep apnea (OSA), are characterized by frequent episodes of upper airway collapse during sleep. These conditions often affect nocturnal sleep quality. Over time, OSA constitutes an independent risk factor for several diseases, including systemic hypertension, cardiovascular disease, stroke, and abnormal glucose metabolism. The estimated prevalence is in the range of 3% to 7%. Diagnostic of sleep apnea and the localization of the site of obstruction requires expensive diagnostic and intervention paradigms, which are only available for a limited number of patients due to the unavailability of sleep laboratories in each hospital.

Sleep studies (polysomnographies) in the clinic or at home can be used to diagnose and grade OSA. For more severe cases of sleep apnea, surgical implantation of a neurostimulator or surgical intervention in the upper airway is considered. Before such a decision, drug-induced sleep endoscopy (DISE) is applied. During a DISE procedure, the patient is anesthetized in an operating room to mimic sleep. At the same time, a small, rigid endoscope is used to determine where the obstruction causing OSA occurs. The procedure is expensive, and it only examines a sleep status that is different from normal sleep to make predictions of how surgical interventions might help. Various traditional OSA diagnosis and treatment options are described in more detail below.

The normal sleep/wake cycle in adults is divided into REM (rapid eye movement) sleep, non-REM (NREM) sleep, and consciousness. NREM sleep is further divided into Stages 1, 2, and 3 NREM sleep. The deepest stage (stage 3 of NREM) is required for the physically restorative effects of sleep, and in pre-adolescents, this is the period of release of human growth hormone. NREM stage 2 and REM, which combined are 70% of an average person's total sleep time, are more associated with mental recovery and maintenance. During REM sleep, in particular, the muscle tone of the throat and neck, as well as the vast majority of all skeletal muscles, is almost completely attenuated. This attenuation allows the tongue and soft palate/oropharynx to relax and, in the case of OSA, to impede the flow of air to a degree ranging from light snoring to complete collapse. When airflow is reduced to a degree where blood oxygen levels fall or the physical exertion to breathe is too great, neurological mechanisms trigger a sudden sleep interruption, called neurological arousal. These arousals rarely result in complete awakening but can significantly negatively affect the restorative quality of sleep. In significant cases of OSA, one consequence is sleep deprivation due to the repetitive disruption and recovery of sleep activity. This sleep interruption in stage 3 (also called slow-wave sleep) and in REM sleep can interfere with normal growth patterns, healing, and immune response, especially in children and young adults.

The diagnosis of OSA is often based on a combination of patient history and tests (in lab or home-based). These tests range, in decreasing order of cost, complexity, and tethering of the patient (number and type of channels of data recorded), from lab-attended full polysomnography (“sleep study”) down to single-channel home recording. In the USA, these categories are associated with insurance classification from Type I to Type IV. In a systematic review of published evidence, the United States Preventive Services Task Force in 2017 concluded that there was uncertainty about the accuracy or clinical utility of all potential screening tools for OSA and recommended that current evidence is insufficient to assess the balance of benefits and harms of screening for OSA in asymptomatic adults.

Polysomnography, when diagnosing OSA, characterizes the pauses in breathing. In OSA, pauses are followed by a relative decrease in blood oxygen; the chest muscles and diaphragm contract, and the entire body may thrash and struggle. An event can be either an apnea, characterized by complete cessation of airflow for at least 10 seconds, or a hypopnea in which airflow decreases by 50 percent for 10 seconds or decreases by 30 percent if there is an associated decrease in oxygen saturation or an arousal from sleep. The number of events per hour is reported as the apnea-hypopnea index (AHI) to grade the severity of OSA. In adults, an AHI of less than 5/h is considered normal. An AHI of 5-15/h is mild, 15-30/h is moderate, and more than 30 events per hour characterize severe OSA.

During drug-induced sleep endoscopy, patients receive sedation administered by an anesthesiologist in the operating room. As patients begin to snore and have some blockage of their breathing, a surgeon passes a flexible telescope through one side of the nose to evaluate the throat and observe the potential blockage of breathing in the palate and tongue regions. There are two major reasons that patients may consider undergoing drug-induced sleep endoscopy. The first is to obtain additional information regarding whether they seem to have blockage of breathing at the palate and/or tongue regions and the second is to determine which specific structures play a major role in airway obstruction. This is true for patients considering surgery for the first time and those who have not obtained ideal results after previous procedures.

Numerous non-surgical treatment options are also used in OSA. It has been recommended to avoid alcohol, smoking, and medications that relax the central nervous system. Weight loss is advised for those who are overweight. Continuous positive airway pressure (CPAP) and mandibular advancement devices are often used and found to be equally effective. Even without weight loss, physical training has been found to improve OSA. There is insufficient evidence to support the widespread use of medications or surgery.

The most widely used current therapeutic intervention is positive airway pressure, whereby a breathing machine pumps a controlled stream of air through a mask worn over the nose, mouth, or both. The additional pressure holds open the relaxed muscles. There are several variants: CPAP is effective for both moderate and severe disease and is the most common treatment for OSA. Automatic positive airway pressure, or “Auto CPAP”, incorporates pressure sensors and monitors the person's breathing. Variable positive airway pressure (VPAP), also known as BiPAP, uses an electronic circuit to monitor the patient's breathing and provides two different pressures: a higher one during inhalation and a lower pressure during exhalation. This system is more expensive and is sometimes used with patients with other coexisting respiratory problems. For example, it is used in patients who find breathing out against an increased pressure uncomfortable or disruptive to their sleep. Nasal EPAP is a bandage-like device placed over the nostrils that utilizes a person's breathing to create positive airway pressure to prevent obstructed breathing.

A 5% reduction in weight among those with moderate to severe OSA may decrease symptoms similarly to CPAP. Also, oral appliances or splints are often preferred but may not be as effective as CPAP. These devices can be in the form of a mouth guard, like those used in sports, to protect the teeth. It is designed to hold the lower jaw slightly down and forward relative to the natural, relaxed position. This position keeps the tongue farther away from the back of the airway and may be enough to relieve apnea or improve breathing. Many people benefit from sleeping at a 30-degree upper body elevation or higher, as if in a recliner. Doing so helps prevent the gravitational collapse of the airway. Sleeping on the side instead of on the back is also recommended.

Surgical treatments to modify airway anatomy, known as sleep surgery, are varied and must be tailored to a patient's specific airway obstruction needs. Surgery is not considered a frontline treatment for OSA in adults, as prospective, randomized, comparative clinical evidence against current front-line treatments is lacking. For those OSA sufferers unable or unwilling to comply with front-line treatment, a properly selected surgical intervention will be the result of considering an individual's specific anatomy and physiology, personal preference, and disease severity. There are a number of different operations that may be performed, including nasal surgery such as turbinectomy (removal or reduction of a nasal turbinate), straightening of the nasal septum or tonsillectomy and adenoidectomy, uvulopalatopharyngoplasty (UPPP) or laser-assisted uvulopalatoplasty (LAUP), genioglossus advancement, hyoid suspension, maxillomandibular advancement, bariatric surgery, etc.

In children, the primary line of treatment is adenotonsillectomy (T&A) surgery. OSA in pediatric patients generally responds to T&A. However, not all children with OSA are surgical candidates and not all patients who undergo T&A surgery for OSA are cured of their OSA. Absence of snoring following surgery does not equal an absence of obstructive apnea. Children with obstructive sleep apnea who are obese generally require follow-up polysomnography 8-12 weeks following adenotonsillectomy to assess for residual sleep apnea and determine whether other interventions are needed. Successful surgical treatment of obstructive sleep apnea is based on the accurate identification of the pattern of airway obstruction and targeted, effective treatment. In the throat, there are two major areas that can be responsible: the palate and tongue regions. For many years, the primary surgical treatment for obstructive sleep apnea was soft palate surgery, and this worked well for patients with blockage of breathing in the palate region alone. Unfortunately, many patients also appear to have blockage of breathing in the tongue region, and multiple procedures have been developed to address this in the hope of improving surgical outcomes.

For patients who cannot use a continuous positive airway pressure device, the U.S. Food and Drug Administration in 2014 granted pre-market approval for an upper airway stimulation system that senses respiration and delivers mild electrical stimulation to the hypoglossal nerve in order to increase muscle tone at the back of the tongue so it will not collapse over the airway. The device includes a handheld patient controller to allow it to be switched on before sleep and is powered by an implantable pulse generator, like one used for cardiac rhythm management. Approval for this active implantable neuromodulation device was preceded by a clinical trial whose results were published. In the market, the device is available with the brand name Inspire Therapy. Inspire therapy is a small, fully implanted system that senses breathing patterns and delivers mild stimulation to maintain multilevel airway patency during sleep. Upper airway stimulation technology provides a first of its kind alternative for those suffering from OSA who are unable to use or get consistent benefit from CPAP. The Inspire system includes three implanted components including a small generator, breathing sensor lead, and stimulation lead, all controlled with a small handheld Inspire sleep remote. Thoughtful patient selection, selective stimulation of targeted airway muscles, and optimal stimulation timing all play an important role in maintaining airway patency and improving patient outcomes.

In contrast to the traditional technologies discussed above, described herein are methods and systems that allow one to identify the site of an obstruction during normal sleep in a home setting. The device includes a small imaging device (e.g., a microcamera) incorporated into a probe, such as a feeding tube. The probe, including the microcamera and a small light source, can be inserted into the upper pharynx of a patient to visually monitor the patient's upper airway. The new device is less invasive than currently available devices, allows for a better assessment of the patient's status, reduces the risk of anesthesia, and reduces health care costs.

In an illustrative embodiment, the proposed device uses a microcamera and one or more small light sources such as light-emitting diodes (LEDs) to monitor the upper airway of a patient during normal daily activities and during sleep. Using the proposed system, drug-induced sleep endoscopy in the operating room will not be necessary anymore. The microcamera and small light sources can be incorporated into a probe formed from small tubing, such as a feeding tube, which can be inserted into the nose, deep enough to capture the upper airway. The camera signal (video footage and/or a series of still images) can be stored on a small portable device in one embodiment. In other embodiments, any type of computing device or computer memory can be used to store captured images/video.

Various helpful features can be extracted from the images and video that are captured by the camera. For example, during natural sleep the system can capture images of an upper airway obstruction during various phases of breathing including snoring, hypopnea, and apnea. The images and video can also capture the site of the obstruction between the sternum and the pharynx. These and other captured features allow the system (or a user of the system) to distinguish between obstructions occurring during exhalation or inhalation. Alternatively, the system can be programmed to identify obstructions that occur during exhalation and during inhalation. Thus, the proposed system relates to diagnosing and characterizing Obstructive Sleep Apnea/Upper airway obstruction. The proposed system also relates to an apparatus, system, and method for noninvasive diagnosis of OSA caused by upper airway obstruction and its treatment.

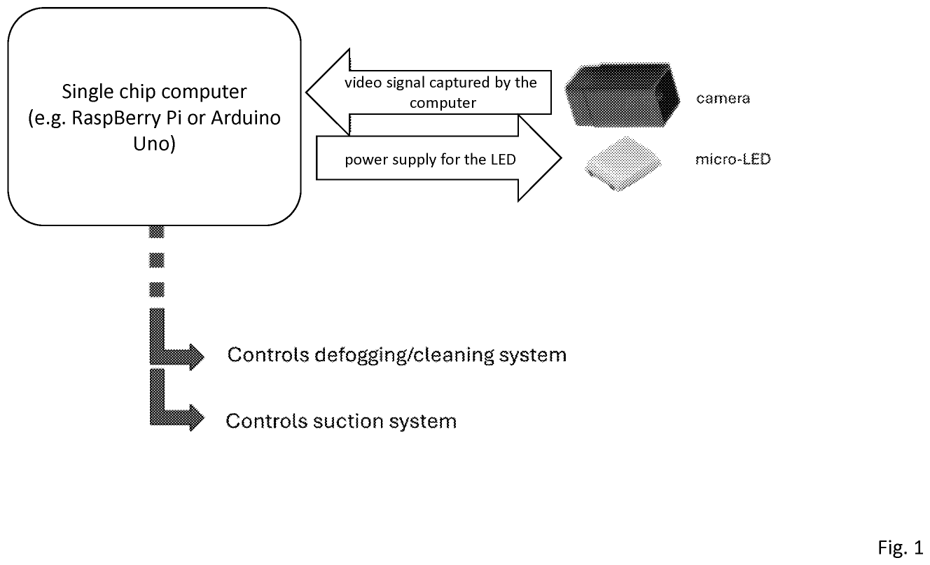

FIG. 1 depicts a schematic of the obstructive sleep apnea diagnosis system in accordance with an illustrative embodiment. As shown in FIG. 1, the system incorporates an imaging device in the form of a microcamera, a light (or illumination) source, and an image post-processing module. In alternative embodiments, the system can include fewer, additional, and/or different components. While the post-processing module is depicted as a single chip computing system, in alternative embodiments a different type of computing device may be used such as a smartphone, laptop computer, desktop computer, cloud computer, etc. The computing system can include a processor, memory, user interface, I/O interface, etc. As discussed in more detail below, the camera and illumination source (e.g., LEDs) can be incorporated into a probe that can be in the form of a tube that also accommodates additional functionality such as suction, defogging/cleaning, etc. The computing system can be used to control the suction, defogging/cleaning functions. The computing system can also control the camera to acquire images/video of a region of interest.

The acquired images are sent to a computer for storage and/or display. In one embodiment, the microcamera used can be the OCHTA10 (Omnivision, California, United States). However, in alternative embodiments, other cameras can be used as well. The OCHTA10 is currently the smallest commercially available camera in the world, with an integrated image array, signal processing, timing, and control circuitry all housed on a single integrated circuit. Its dimensions are compact at 0.65×0.65 mm, with a z-height of 1.19 mm, and the camera offers a resolution of 400×400 pixels. The camera can capture high-quality images and video up to 30 frames per second. Despite its miniature size, the image sensor incorporates advanced imaging technology, including micro-lenses. Furthermore, the camera chip is designed to be power-efficient, boasting a low power consumption of just 25 milli-Watts (mW). In one embodiment, the illumination source can include micro-LEDs models emitting yellow light (λ=591 nm), each with a different size. For example, the micro-LEDs can include the model 0402 from Evemodel (China), with dimensions of 1.0×0.5×0.40 mm, the Nanopoint 0201 by SunLED (California, United States), which measures 0.65×0.35×0.2 mm³ and is currently the smallest available micro-LED on the market, etc. Alternatively, other types of LEDs or other types of light sources may be used.

The computing module for post-processing the images captured by the microcamera OCHTA10 can include two main components: a video bridge chip, specifically the OAH0428 (Omnivision, California, United States), and a digital signal processor (DSP). The OAH0428 is mainly chosen for its compatibility with the OCHTA10, offering integrated analog-to-digital conversion and a digital video parallel output (DVP). Alternatively, a different type of video bridge and/or DSP may be used. In an illustrative embodiment, the video bridge chip converts the camera's analog video signals into a digital format. After this initial processing, the DSP further processes the digital signals. The resulting video data, now fully processed, can be transmitted to the computer via a USB connection. Alternatively, a different type of wired or wireless connection (e.g., Bluetooth) may be used for transmission. For the final step, the data is visualized using (e.g., Amcap) software, allowing for a detailed examination and analysis of the images captured by the camera.

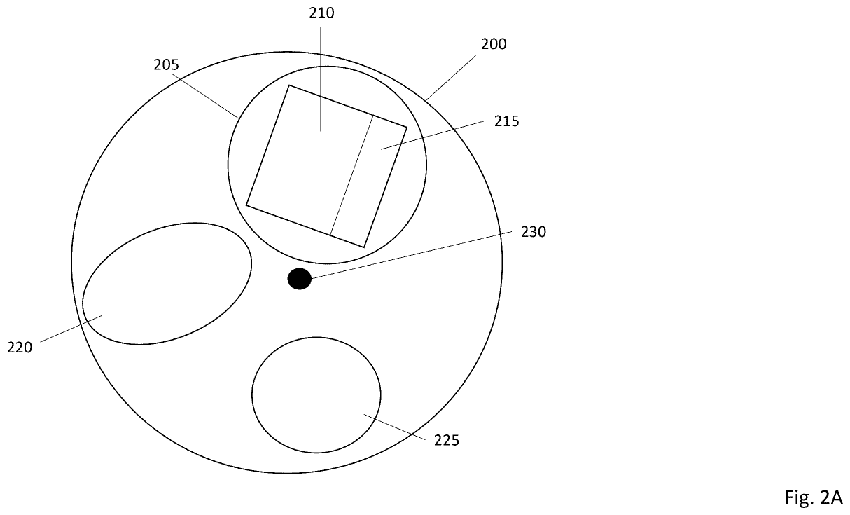

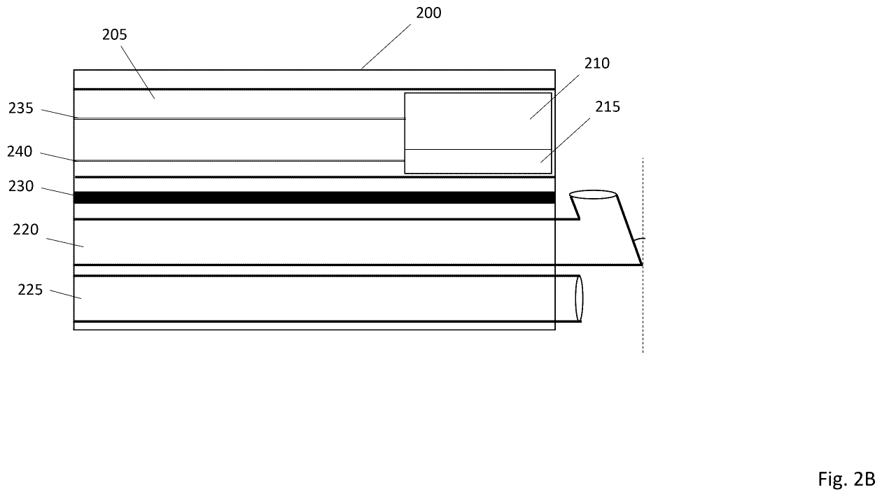

In an illustrative embodiment, the proposed imaging system is incorporated into a probe (tube system). In one embodiment, the probe can be a modified feeding tube. Alternatively, the probe can be a custom component, a length and diameter of which are sized according to the patient's airway. FIG. 2A is an end view of a probe for performing obstructive sleep apnea diagnosis in accordance with an illustrative embodiment. FIG. 2B is a cross-sectional side view of the probe for diagnosing obstructive sleep apnea in accordance with an illustrative embodiment. As shown, the probe 200 includes a first port (imaging port) 205 that includes an imaging device 210 and an illumination source 215. The probe 200 also includes a second port (irrigation port) 220 that is sized to provide irrigation (e.g., air, water, saline solution, etc.) to the system from an irrigation source. The irrigation can be used to move obstructions, to clean a lens of the imaging device, to clean the illumination system, to clean the region of interest, etc. The probe 200 further includes a third port (vacuum port) 225 that is sized to provide suction to the system for cleaning of the region of interest, removal of debris, etc. The third port 225 is connected to a vacuum source. The probe 200 also includes a stabilization wire 230 that is used to maintain the (flexible) probe in a desired shape or configuration.

As shown in FIG. 2B, a distal end of the irrigation port 220 is angled toward the first port such that irrigation (air or liquid) can be used to clean a lens of the imaging system 210 and/or the illumination system 215. As such, in the event that blood, tissue, or debris blocks the view of the imaging device 210, the user can control an irrigation source so that liquid or air travels through the irrigation port 220 and cleans the imaging device. As a result, the system can be cleaned for continued use without having to remove the device for cleaning, and then replace it, which causes additional stress and risk to the patient. As shown, the distal end of the irrigation port 220 forms an angle (see angle formed relative to the vertical dashed line) as it extends from the distal end of the probe. This angle can be 20 degrees in one embodiment. Alternatively, a different angle can be used such as 15 degrees, 30 degrees, 45 degrees, etc.

As shown in the side view of FIG. 2B, wires 235 are used to connect the imaging device 210 to a computing system. The wires 235 can be used to deliver power to the imaging device from a power source (e.g., battery, wall outlet, etc.), to deliver control signals that control operation of the image device 210, to receive image/video data from the imaging device 210, etc. The system also includes wires 240 that are used to connect the illumination source 215 to the computing system. The wires 240 can be used to deliver power to the illumination source 215, control signals to control operation of the illumination source 215, etc. In an alternative embodiment, the wires 235 and/or the wires 240 may not be used. In such an embodiment, one or more power sources can be incorporated into the imaging device 210 and/or the illumination source 215. Additionally, in such an embodiment, control signals and/or captured images/video can be transmitted to/from the computing system using wireless communication.

In one embodiment, the probe 200 can be made from a feeding tube that is modified to incorporate the various ports. Alternatively, the probe can be a custom device that is formed to include the ports and other features as shown in FIG. 2. The probe can be formed via three-dimensional (3D) printing, molding, or any other manufacturing technique, and can be made from any biocompatible material such as plastic, rubber, silicon, etc.

In use, the probe can be inserted through one nostril of a patient. During the insertion process, the tube passes through the pharynx. The illumination source and the imaging device contained within a port of the probe allows the operator to carefully monitor the insertion of the probe and avoid tissue damage. To monitor the pharynx and upper airway, the probe insertion is stopped and the probe can be secured at the patient's skin. The camera then can be used to capture video and/or still images to monitor footage of the upper airway behavior during sleep and/or periods of being awake. The captured material is analyzed to identify sites of airway obstruction during daily life and during sleep.

To form the proposed system, in one embodiment, the imaging device (e.g., camera) is inserted into the probe such that the camera's optical system rests at the opening of the tube. To provide illumination for the microcamera, the illumination source is strategically attached laterally to the imaging system. The placement is such that the smaller dimension of the micro-LED (or other light source) contributes to a minimal increase in the camera assembly's overall width, ensuring the design's compactness. To secure the micro-LEDs firmly and maintain the assembly's integrity, a UV-curable polymer OrmoComp® can be used. Alternatively, a different type of polymer or adhesive may be used. The imaging device and/or illumination source can also be secured with medical grade silicone in the tubing in one embodiment. In operation, the camera connects to a computer device (e.g., RaspBerry Pi, Arduino Uno, smartphone, laptop, etc.), to continuously record the captured images and video footage. The image/video files can be transferred from the camera by a wired connection or wirelessly through the internet.

In at least some embodiments, forming the system also includes forming the probe to include (or be sized to receive) an irrigation port. In one embodiment, the irrigation port can include a universal connector that connects to an irrigation source, such as a container of liquid or compressed gas. As discussed, an end of the irrigation port can be angled toward the imaging port to assist with lens cleaning. Forming the system can further include forming the probe to include (or be sized to receive) a vacuum port. In one embodiment, the vacuum port can include a universal connector that connects to a vacuum source such that the vacuum port can be used to suck out blood and other debris from so that the region of interest can be better visualized. The universal connectors can be valves in one embodiment that enable on/off and/or gradual control of the irrigation source and the vacuum source. In operation, both the irrigation source (or its associated valve) and the vacuum source (or its associated valve) can be connected to and controlled by the computing system.

Thus, in contrast to current technology, the proposed methods and systems will allow one to identify the site of an obstruction during normal sleep in a home setting. In one embodiment, the system includes a small camera and accompanying light source incorporated into a probe, which can be a feeding tube. In use, the camera and the small light source are inserted into the upper pharynx of a patient to monitor the patient's upper airway. In some embodiments, the probe can also include an irrigation port connected to an irrigation source (e.g., liquid, compressed air, etc.) for cleaning the observation area and/or the camera via air or liquid to improve the overall visualization without having to remove and reinsert the system. The probe can also include a vacuum port connected to a vacuum source that can be used to suck up and remove blood, tissue, or other debris that inhibit visualization of the region of interest. The new device is less invasive, allows for a better assessment of the patient's status, reduces the risk of anesthesia, and reduces health care costs when compared to currently available systems.

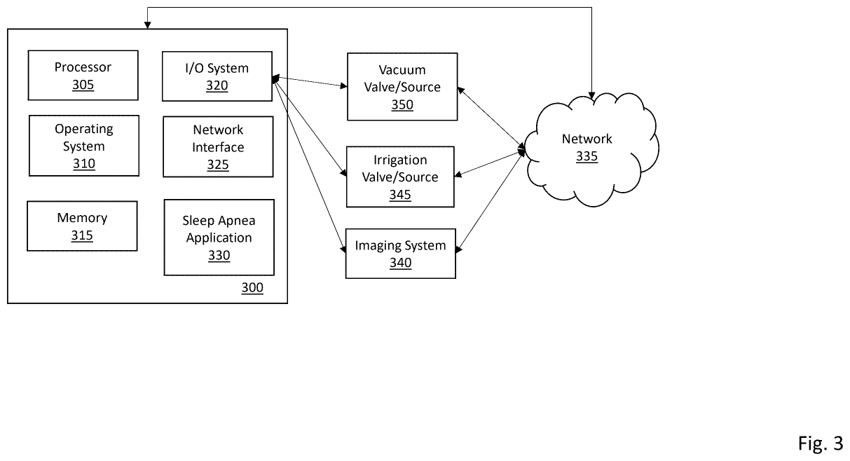

As discussed above, any of the operations described herein can be performed by a computing system that includes a memory, processor, user interface, network interface, display, etc. For example, any of the operations described herein can be implemented as computer-readable instructions stored on a computer-readable medium. Upon execution of the computer-readable instructions by the processor, the computing system performs the various operations described herein to implement the system. As an example, FIG. 3 is a block diagram of a computing system 300 to implement the sleep apnea monitoring system in accordance with an illustrative embodiment. The computing system 300 is in communication with an imaging system 340, an irrigation valve/source 345, and a vacuum valve/source 350, either directly, or wirelessly through a network 335. The imaging system 340 can be any type of camera or imager that is able to capture events as described herein, and can include a light source as described herein. In one embodiment, the imaging system 340 can include its own processor, memory, transceiver, user interface, etc. in addition to the image capturing components, such as one or more lenses, etc. The imaging system 340 can be used to monitor and capture image data that can be used to determine the presence and location of sleep apnea.

The computing system 300 includes a processor 305, an operating system 310, a memory 315, an input/output (I/O) system 320, a network interface 325, and a sleep apnea application 330. In alternative embodiments, the computing system 300 may include fewer, additional, and/or different components. The components of the computing system 300 communicate with one another via one or more buses or any other interconnect system. The computing system 300 can be any type of computing device (e.g., tablet, laptop, desktop, etc.) that has sufficient processing power to perform the operations described herein.

The processor 305 can be in electrical communication with and used to control any of the system components described herein. For example, the processor can be used to execute the sleep apnea application 330, process received user selections, send data and commands to the imaging system 340, irrigation valve/source 345, and vacuum valve/source 350, receive raw data from the imaging system 340, process the data using algorithms, etc. The processor 305 can be any type of computer processor known in the art, and can include a plurality of processors and/or a plurality of processing cores. The processor 305 can include a controller, a microcontroller, an audio processor, a graphics processing unit, a hardware accelerator, a digital signal processor, etc. Additionally, the processor 305 may be implemented as a complex instruction set computer processor, a reduced instruction set computer processor, an x86 instruction set computer processor, etc. The processor 305 is used to run the operating system 310, which can be any type of operating system.

The operating system 310 is stored in the memory 315, which is also used to store programs, user data, network and communications data, peripheral component data, the sleep apnea application 330, and other operating instructions. The memory 315 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 315 can be in the cloud to provide cloud storage for the system. Similarly, in one embodiment, any of the computing components described herein (e.g., the processor 305, etc.) can be implemented in the cloud such that the system can be run and controlled through cloud computing.

The I/O system 320 is the framework which enables users and peripheral devices to interact with the computing system 300. The I/O system 320 can include a display to show captured images/video, one or more speakers, one or more microphones, a keyboard, a mouse, one or more buttons or other controls, etc. that allow the user to interact with and control the computing system 300. The I/O system 320 also includes circuitry and a bus structure to interface with peripheral computing devices such as power sources, universal service bus (USB) devices, data acquisition cards, peripheral component interconnect express (PCIe) devices, serial advanced technology attachment (SATA) devices, high definition multimedia interface (HDMI) devices, proprietary connection devices, etc.

The network interface 325 includes transceiver circuitry (e.g., a transmitter and a receiver) that allows the computing system 300 to transmit and receive data to/from other devices such as the imaging system 340, the irrigation valve/source 345, the vacuum valve/source, other remote computing systems, servers, websites, etc. The network interface 325 enables communication through the network 335, which can be one or more communication networks. The network 335 can include a cable network, a fiber network, a cellular network, a wi-fi network, a landline telephone network, a microwave network, a satellite network, etc. The network interface 325 also includes circuitry to allow device-to-device communication such as Bluetooth® communication.

The sleep apnea application 330 can include software and algorithms in the form of computer-readable instructions which, upon execution by the processor 305, performs any of the various operations described herein such as capturing images using the imaging system 340, controlling the irrigation valve/source 345 such that air or fluid is released into the probe for removing debris from the imaging system 340, controlling the vacuum valve/source 350 to remove debris from the imaging system 340, processing captured image data, determining whether sleep apnea is present, determining a location of the sleep apnea, etc. The sleep apnea application 330 can utilize the processor 305 and/or the memory 315 as discussed above. In an alternative implementation, the sleep apnea application 330 can be remote or independent from the computing system 300, but in communication therewith.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of diagnosing obstructive sleep apnea, the method comprising:
mounting a camera in a flexible probe that is sized to fit within a nasal cavity of a patient;
capturing, by the camera mounted within the probe, images of the patient while the patient is sleeping; and
analyzing, by a computing system in communication with the camera, the captured images to determine whether sleep apnea is present.

2. The method of claim 1, wherein the flexible probe includes a stabilization wire that bends to conform the flexible probe to the nasal cavity of the patient.

3. The method of claim 1, further comprising analyzing the captured images to identify a location of the sleep apnea.

4. The method of claim 1, wherein the camera is mounted in an imaging port of the flexible probe, wherein the flexible probe also includes an irrigation port, and further comprising controlling, by the computing system, an irrigation source that is connected to the irrigation port.

5. The method of claim 4, wherein an end of the irrigation port is angled toward the imaging port, and further comprising controlling the irrigation source to clean the camera.

6. The method of claim 1, wherein the camera is mounted in an imaging port of the flexible probe, wherein the flexible probe also includes a vacuum port, and further comprising controlling, by the computing system, a vacuum source that is connected to the vacuum port.

7. The method of claim 6, wherein controlling the vacuum source comprises activating the vacuum source to remove debris from an end of the flexible probe such that the debris flows through the vacuum port.

8. The method of claim 1, further comprising activating, by the computing system, a light source for the camera to provide illumination such that the images can be captured.

9. The method of claim 1, wherein the flexible probe is positioned in in an upper pharynx of the patient to monitor an upper airway of the patient.

10. The method of claim 1, further comprising determining, by the computing system, whether an obstruction relating to the sleep apnea occurs during inhalation or during exhalation.

11. A system for use in diagnosis of obstructive sleep apnea, the system comprising:
a probe that includes an imaging port;
a camera sized to fit into the imaging port; and
an illumination source sized to fit into the imaging port alongside the camera;
wherein an outer diameter of the probe is sized to fit within a nostril of a patient such that the camera captures images of a portion of an airway of the patient.

12. The system of claim 11, wherein the probe further includes an irrigation port that connects to an irrigation source.

13. The system of claim 12, wherein the irrigation port includes a universal connector that attaches to the irrigation source.

14. The system of claim 13, wherein the universal connector comprises a valve that controls the irrigation source.

15. The system of claim 12, wherein a distal end of the irrigation port is angled toward a distal end of the imaging port.

16. The system of claim 12, wherein a distal end of the imaging port is flush with a distal end of the probe, and wherein a distal end of the irrigation port extends past the distal end of the probe.

17. The system of claim 11, wherein the probe further includes a vacuum port that connects to a vacuum source.

18. The system of claim 17, wherein the vacuum port includes a universal connector that connects to the vacuum source.

19. The system of claim 11, wherein the illumination source comprises a plurality of light-emitting diodes.

20. The system of claim 11, further comprising a stabilization wire within the probe, wherein the stabilization wire maintains the probe in a desired shape.
